SEQUENCE LISTING (4 sequences)

SEQ ID NO: 1 - DNA, 609 bp, Artificial Sequence
  Nucleic acid sequence - full length native Recoverin
atggggaacagcaagagtggggccctgtccaaggagatcctggaggagctgcagctgaacaccaagttcacggaggaggagctgagctcctggtaccagtccttcctgaaggagtgccccagtggccggatcacccggcaggagttccagaccatctactccaagttcttccccgaggccgaccccaaggcctatgcccagcacgtgttccgaagctttgatgccaacagcgatggcaccttggacttcaaggagtatgtcatcgccctacacatgaccagcgcgggcaagaccaaccagaagctggagtgggccttctccctctatgatgtggatggcaatgggaccatcagcaagaacgaggtgctggagattgtcacggctatcttcaaaatgatcagccctgaggacacaaagcatctcccagaagacgagaacactccggaaaagcgagcagagaagatctggggattctttggcaagaaggatgatgataaacttacagagaaagaattcatcgaagggaccctggccaataaggaaattctgcgactgattcaattcgagcctcaaaaagtgaaggagaaactgaaggaaaagaaactctga

SEQ ID NO: 2 - protein, 201 aa, Artificial Sequence
  Amino acid sequence - full length native Recoverin (start Met is removed)
GNSKSGALSKEILEELQLNTKFTEEELSSWYQSFLKECPSGRITRQEFQTIYSKFFPEADPKAYAQHVFRSFDANSDGTLDFKEYVIALHMTSAGKTNQKLEWAFSLYDVDGNGTISKNEVLEIVTAIFKMISPEDTKHLPEDENTPEKRAEKIWGFFGKKDDDKLTEKEFIEGTLANKEILRLIQFEPQKVKEKLKEKKL

SEQ ID NO: 3 - DNA, 642 bp, Artificial Sequence
  Nucleic acid sequence - mutated Recoverin (+ NdeI and BamHI restriction sites, + silent mutation, without myristoyl group)
  misc_feature (1)..(6): Nde1 site (CATATG)
  misc_feature (333)..(333): T to C silent mutation
  misc_feature (610)..(636): 5 glycines (GGC or GGT) at the end  + thrombin cleavage site [Leu-Val-Pro-Arg-Gly-Ser  L (CTG), V (GTT), P (CCG), R (CGT), G (GGA), S (TCC)]
  misc_feature (637)..(642): Stop codon removed and replaced by BamH1 site (GGATTC) that corresponds also to glycine and serine from thrombin recognition site
catatggggaacagcaagagtggggccctgtccaaggagatcctggaggagctgcagctgaacaccaagttcacggaggaggagctgagctcctggtaccagtccttcctgaaggagtgccccagtggccggatcacccggcaggagttccagaccatctactccaagttcttccccgaggccgaccccaaggcctatgcccagcacgtgttccgaagctttgatgccaacagcgatggcaccttggacttcaaggagtatgtcatcgccctacacatgaccagcgcgggcaagaccaaccagaagctggagtgggccttctccctctatgacgtggatggcaatgggaccatcagcaagaacgaggtgctggagattgtcacggctatcttcaaaatgatcagccctgaggacacaaagcatctcccagaagacgagaacactccggaaaagcgagcagagaagatctggggattctttggcaagaaggatgatgataaacttacagagaaagagttcatcgaagggaccctggccaataaggaaattctgcgactgattcaattcgagcctcaaaaagtgaaggagaaactgaaggaaaagaaactcggcggtggtggcggcctggttccgcgtggatcc

SEQ ID NO: 4 - protein, 212 aa, Artificial Sequence
  Amino acid sequence - mutated Recoverin (+ NdeI and BamHI restriction sites, - EcoR1 silent mutation)
  MISC_FEATURE (202)..(212): Thrombin cleavage site
GNSKSGALSKEILEELQLNTKFTEEELSSWYQSFLKECPSGRITRQEFQTIYSKFFPEADPKAYAQHVFRSFDANSDGTLDFKEYVIALHMTSAGKTNQKLEWAFSLYDVDGNGTISKNEVLEIVTAIFKMISPEDTKHLPEDENTPEKRAEKIWGFFGKKDDDKLTEKEFIEGTLANKEILRLIQFEPQKVKEKLKEKKLGGGGGLVPRGS

(19) United States
(12) Patent Application Publication
(10) Pub. No.: US 2018/0208634 A1
(43) Pub. Date: Jul. 26, 2018

(54) Recoverin as a Fusion Protein Tag to Improve Expression, Solubility and Purification of Proteins

(71) Applicant: UNIVERSITÉ LAVAL, Quebec (CA)

(72) Inventors: Christian Salesse, Quebec (CA); Line Cantin, Quebec (CA)

(73) Assignee: UNIVERSITE LAVAL, Quebec (CA)

(21) Appl. No.: 15/746,540

(22) PCT Filed: Jul. 19, 2016
(86) PCT No.: PCT/CA2016/050849

§ 371 Date: Jan. 22, 2018

Related U.S. Application Data
(60) Provisional application No. 62/194,348, filed on Jul. 20, 2015.

(51) Int. Cl. C07K 14/47; C12N 9/10; C07K 1/22

(52) U.S. Cl. CPC C07K 14/4728; C12N 9/1029; C07K 2319/20; C07K 1/22; C12Y 203/01135

(57) ABSTRACT

Herein presented is a new, versatile fusion protein tool (tag) to improve the solubility and purification of proteins. Particularly, the fusion tag is the substantially full-length (about 23 kD) Recoverin molecule (TagR) that is used for protein purification in a single step, even in the presence of detergents.

### FIELD

The present invention relates to a new, versatile fusion protein tool (tag) to improve the expression, solubility and purification of proteins. Particularly, the fusion tag is the substantially full-length Recoverin molecule, as well as the other members of EF-hand family of neuronal calcium sensor proteins, which bind calcium, that can be used for protein purification in a single step, even in the presence of detergents.

### BACKGROUND

Because of the availability of the genome sequence of a large number of organisms, recombinant techniques have allowed the identification, modification, production, and purification of a large number of proteins using different host cell types¹⁻¹³. Consequently, there has been a large increase in the use of recombinant proteins for a variety of applications including therapeutic and diagnostic uses.² In addition, numerous laboratories are expressing novel proteins to determine their structure and function, or to discover their therapeutic potential. As well, pharmaceutical companies are involved in large scale production of proteins with therapeutic purposes. High protein purity is thus necessary to satisfy these requirements. It is however very difficult to purify proteins using their intrinsic properties. The use of fusion tags is therefore widely spread to facilitate protein purification.

The production of recombinant proteins often results in the formation of inclusion bodies, host cell toxicity, low levels of expression or improper folding of proteins.² These problems can often be solved by changing the expression system, the host cell type or by using a fusion tag.² Many fusion tags are available¹¹ and new tags or new tagging procedures are continuously provided.¹⁴⁻¹⁷ These tags can be located at either the N- or C-terminus of the protein of interest, but the choice of the most appropriate tag depends on each protein's particular properties.

The most commonly used small affinity tag is the polyhistidine tag (His-tag). It allows purification of proteins by immobilized metal affinity chromatography. It is typically made of hexahistidine although decahistidine is often more efficient for protein purification in a single step. However, this tag does not allow for improvement of the yield and increased solubility of the protein of interest. Therefore, the most widely used protein tag is the Glutathione S-transferase (GST),¹⁸ that is considered as the «Gold Standard». It is a 26 kDa protein which binds with high affinity to glutathione. GST can increase the yield and solubility of proteins of interest, and purification is achieved by binding of GST-tagged proteins to glutathione immobilized to a solid support.

Although the GST tag was found to be very useful to improve the solubility and purification of several proteins of interest, it has several drawbacks. For example, purification of GST-tagged proteins cannot be achieved in the presence of detergents.¹⁹ Moreover, separation of the protein of interest from GST after their cleavage is difficult to readily achieve because of the high affinity of glutathione for GST, which consequently requires an additional step of dialysis, unless if cleavage can be performed on the column. New protein tags are thus needed to improve solubility and facilitate purification of proteins of interest.

### SUMMARY

According to a first aspect, there is provided a polynucleotide molecule comprising a sequence encoding a protein of interest to be purified, and a tag polynucleotide encoding a protein of an EF-hand calcium-binding family of proteins, wherein the EF-hand calcium-binding protein undergoes conformational change under presence or absence of calcium. Particularly, the conformational change comprises an extrusion of hydrophobic amino acids in the presence of calcium. More particularly, the tag is a member of the EF-hand calcium-binding family of neuronal calcium sensor proteins.

In accordance with a further particular aspect, the invention provides for a vector comprising the polynucleotide as defined herein, operably linked to a promoter.

In accordance with a particular aspect of the invention there is provided an expression cassette comprising: a transcriptional initiation region functional in an expression host cell; the recombinant polynucleotide as defined herein; and a transcriptional termination region functional in the expression host cell.

In accordance with a particular aspect of the invention, there is provided a host cell comprising the expression cassette as defined herein.

In accordance with a particular aspect of the invention, there is provided a method for expressing a protein of interest, comprising the steps of: expressing the protein of interest fused to a Recoverin tag (TagR) in an expression system comprising the vector, or the expression cassette as defined herein.

In accordance with a particular aspect of the invention, there is provided a method of expression of a protein of interest, comprising the steps of: expressing the protein of interest fused to a Recoverin tag (TagR) in a host cell as defined herein. According to a more particular aspect of the invention of this method, the expression of the protein is carried out with minimal growth media to allow folding of the tag protein.

In accordance with a particular aspect of the invention, there is provided a method for producing a protein of interest, wherein the method comprises: growing a cell under conditions that permit expression of the protein as defined herein, wherein the cell comprises: the polynucleotide; or the vector; or the expression cassette, all as defined herein.

In accordance with a particular aspect of the invention, there is provided a method for purifying a protein of interest, comprising the steps of: expressing the protein of interest as defined herein; or producing the protein of interest as defined herein; and purifying the fused-protein by separating the fused protein from unwanted components on hydrophobic affinity chromatography in presence of calcium. Particularly, the purifying step is carried out by eluting the fused-protein from the hydrophobic affinity column in presence of a calcium chelator; and optionally cleaving the eluted fused molecule in order to obtain the purified protein of interest cleaved from the tag. Alternatively, the purifying step is carried out by cleaving the fused-protein of interest from the tag to obtain a mixture comprising the cleaved protein and the tag, and separating the cleaved protein of interest from the tag by eluting the cleaved protein on the hydrophobic affinity column in presence of a calcium chelator.

In accordance with a particular aspect of the invention, there is provided a fusion protein comprising a protein of interest, fused to a protein of an EF-hand calcium-binding family of proteins, wherein the EF-hand calcium-binding protein undergoes conformational change under presence or absence of calcium.

In accordance with a particular aspect of the invention, there is provided a fusion protein encoded by the polynucleotide as defined herein. According to a particular aspect, the encoded protein tag is nonmyristoylated.

In accordance with a particular aspect of the invention, there is provided a kit for the expression and purification of a protein of interest, the kit comprising: the polynucleotide as defined herein; and instructions on how to insert the polynucleotide in a suitable vector; and/or instructions on how to transform the vector in a host cell; and/or instructions on how to isolate a recombinant fused-protein from the host cell; and/or instructions on how to purify the recombinant fused-protein as defined herein.

In accordance with a particular aspect of the invention, there is provided a kit for the expression and purification of a protein of interest, the kit comprising: the vector, or the expression cassette, both as defined herein; and instructions on how to transfect the vector in a host cell; and/or instructions on how to isolate a recombinant fused-protein from the host cell; and/or instructions on how to purify the recombinant fused-protein according to the method as defined herein.

According to a particular aspect of the invention, there is provided use of a protein from an EF-hand calcium-binding family defined herein, as a tag for protein expression and/or solubilisation and/or purification.

In accordance with a particular aspect of the invention, there is provided a protein from an EF-hand calcium-binding family defined herein for use as a protein tag for protein expression and/or solubilisation and/or purification.

### DESCRIPTION

### Description of the Figures

FIG. 1. A large conformational change of Recoverin (TagR) is induced by the binding of calcium. A) The binding of calcium by two of its four EF-hands induces the extrusion of its myristoyl group and of several hydrophobic amino acids. B) The myristoyl group of TagR and several hydrophobic residues are sequestered in a hydrophobic pocket in the absence of calcium (in the presence of EGTA). This large conformational change allows purification of TagR using hydrophobic chromatography. Indeed, the TagR binds the hydrophobic resin in the presence of calcium (A) which allows removal of the contaminating proteins by extensively washing the column. The addition of EGTA (B) allows sequestering the hydrophobic amino acids inside TagR and its elution from the column. This property was shown to allow achieving high purity of this protein.₄₂

FIG. 2. Comparison between the expression level of TagR-tLRAT at 21° C. for 16 h in the pGEX 4T-3 and pET11a vectors. Lane 1, pGEX-4T-3. Lane 2, pET11a. Lane «a», molecular mass standards.

FIG. 3. Comparison between the expression level of TagR-tLRAT and GST-tLRAT at 21° C. for 16 h in the pGEX-4T-3 vector. Lane 1, TagR-tLRAT. Lane 2, GST-tLRAT. Lane «a», molecular mass standards.

FIG. 4. Comparison between the solubility of TagR-RP2 in pET11a and that of GST-RP2 in pGEX-4T-3. Lanes 1, 2 and 3, TagR-RP2; Lanes 4, 5 and 6, GST-RP2; Lanes 1 and 5, total lysate. Lanes 2 and 4, supernatant after centrifugation of total lysate at 20,000×g for 30 min at 4° C. Lanes 3 and 6, pellet after centrifugation. Lane «a», molecular mass standards.

FIG. 5A. Comparison between the solubility of TagR-tLRAT (lanes 1, 3 and 5) and that of GST-tLRAT (lanes 2, 4 and 6) in pGEX-4T-3. Lanes 1 and 2, total lysate. Lanes 3 and 4, supernatant after centrifugation of total lysate at 20,000×g for 30 min at 4° C. Lanes 5 and 6, pellet after centrifugation. Lane «a», molecular mass standards.

FIG. 5B. Comparison between the expression level and solubility of TagR-tLRAT at 21° C. for 16 h in the LB (lanes 1 to 3) and minimal (lanes 4 to 6) growth media. Lanes 1 and 4, total lysate. Lanes 2 and 5, supernatant after a centrifugation at 20,000×g for 30 min at 4° C. Lanes 3 and 6, pellet. Arrows are showing the position of TagR-tLRAT. Lane «a», molecular mass standards.

FIG. 6. Purification and cleavage of TagR-RP2 and separation of RP2 from its cleaved TagR fusion partner. Lane 1, proteins which did not bind the column. Lane 2, extensive wash of the column in the presence of calcium. Lane 3, elution of highly purified TagR-RP2 in a single step in the absence of calcium (in the presence of EGTA). Lane 4, quantitative cleavage of RP2 from TagR using the protease thrombin. Lane 5, elution of RP2 in the presence of calcium after loading the cleaved sample on the same column (phenyl Sepharose). Lane 6, elution of TagR in the presence of EGTA. Lanes «a», molecular mass standards. Cleavage of TagR-RP2 can also be performed on the column. Thrombin is eliminated by connecting a Hitrap Benzamidine FF sepharose column to that of phenyl Sepharose.

FIG. 7. Purification and cleavage of TagR-tLRAT and purification of tLRAT from its cleaved TagR fusion partner. Lane 1, total bacteria lysate (the arrow is showing the band corresponding to tLRAT) using bacteria cultured in a minimal growth medium. Lane 2, supernatant after centrifugation of the total lysate (soluble fraction). Lane 3, pellet after centrifugation (insoluble fraction). Lane 4, proteins which did not bind the column. Lane 5, washing of the column. Lane 6, elution of highly purified TagR-tLRAT in a single step in the absence of calcium (in the presence of EGTA). The result of the cleavage of TagR-tLRAT using thrombin for 36h at 4° C. is not shown. Lane 7, Elution of TagR in the presence of calcium after loading the cleaved sample on the same column. Lane 8, elution of tLRAT using pure water. Lanes «a», molecular mass standards. It must be stressed that the electrophorosis gel showing purification of TagR-tLRAT (lane 6) contains 12% acrylamide compared to 15% for the other gels: the molecular mass of TagR-tLRAT in lanes 1 and 6 (see arrows) is thus the same on the basis of their respective molecular mass standards (lanes «a»).

FIG. 8. Binding of TagR-tLRAT to the hydrophobic phenyl sepharose column in the presence of SDS and purification of the fusion protein. Lane 1, bacteria lysate. Lane 2, supernatant after centrifugation (soluble fraction). Lane 3, Pellet after centrifugation (insoluble fraction). Lane 4, proteins which did not bind to the column. Lane 5, washing of the column with buffer A containing no SDS. Lane 6, elution of the highly purified TagR-tLRAT in a single step in the presence of EGTA, similarly to the data shown in FIG. 7 (lane 6). Lane «a», molecular mass standards.

FIG. 9. Purification of PolyHis-TagR-tLRAT in the presence of SDS on a nickel resin, cleavage of PolyHis-TagR and purification of tLRAT. Lane 1, bacteria lysate. Lane 2, supernatant after centrifugation (soluble fraction). Lane 3, proteins which did not bind the column. An extensive washing of the column was performed to remove any trace of SDS as it prevents the action of thrombin. Cleavage of PolyHis-TagR-tLRAT directly on the column using thrombin for 40 h. Lane 4, elution of pure tLRAT using 0.1% SDS (see arrow). Lane 5, elution of uncleaved PolyHis-TagR-tLRAT together with the cleaved PolyHis-TagR using 150 mM imidazole. Lanes «a», molecular mass standards.

### ABBREVIATIONS AND DEFINITIONS

### Abbreviation

BAPTA: 1,2-Bis(2-Aminophenoxy)ethane-N,N,N′,N′-tetraacetic acid; EDTA: Ethylenediaminetetraacetic acid; EGTA: Ethylene glycol-bis(2-aminoethylether)-N,N,N′,N′-tetraacetic acid; tLRAT: truncated lecithin retinol acyltransferase; RP2: retinitis pigmentosa 2; and TagR: Recoverin protein tag.

GCAP: guanylate cyclase activating protein; GCIP: guanylate cyclase inhibiting protein; KChIP: potassium channel interacting protein; NCS1: neuronal calcium sensor 1; VILIP-1: Visinin-like protein.

TEV: Tobacco Etch Virus; HRV 3C: Human rhinovirus 3C.

### Definitions

The term “about” as used herein refers to a margin of + or −10% of the number indicated. For sake of precision, the term about when used in conjunction with, for example: 90% means 90%+/−9% i.e. from 81% to 99%. More precisely, the term about refer to + or −5% of the number indicated, where for example: 90% means 90%+/−4.5% i.e. from 86.5% to 94.5%.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

The term “gene” as used herein refers to any DNA sequence comprising several operably linked DNA fragments such as a promoter and a 5′ regulatory region, a coding sequence and an untranslated 3′ region comprising a polyadenylation site.

The term “expression cassette” as used herein refers to a transferable region of DNA comprising a chimeric gene which is flanked by one or more restriction or other sites which facilitate precise excision from one DNA locus and insertion into another.

The term “protein of interest” as used herein means any protein for which small or large scale production may present an interest for a person skilled in the art.

### Detailed Description of Particular Embodiments

Recoverin (full-length or substantially full-length) has now been found to possess properties suitable for protein tags. Recoverin has a molecular weight of about 23 kDa, is highly soluble (>30 mg/ml), it can be highly purified in a single step and its expression level in bacteria is very high (>30 mg/L of culture).²⁰ It is predicted that, based upon the similar function and properties of this family of proteins, other members of EF-hand family of neuronal calcium sensor proteins, which bind calcium, will also behave as suitable protein tags for purification.

### Other Protein Tags

In addition to Recoverin, the neuronal calcium sensor family of proteins include GCAP1, GCAP2 or GCAP3 (guanylate cyclase activating protein), GCIP (guanylate cyclase inhibiting protein), KChIP1, KChIP2 (potassium channel interacting protein), Calsenilin/DREAM, NCS1/Frequenin (neuronal calcium sensor 1), Neurocalcin delta, Hippocalcin, VILIP-1 (Visinin-like protein). There is a large amino acid sequence identity between the different members of this family of proteins. For example, there is 61% identity between Frequenin and Neurocalcin delta and 41% between Frequenin and Recoverin⁶¹.

Therefore, in accordance with a particular aspect of the invention there is provided the polynucleotide as defined herein, wherein the encoded tag protein comprises at least about 40% identity with Recoverin, more particularly, the encoded tag protein comprises at least about 60% identity with Recoverin.

Similarly, there is a high similarity between the overall structure of the members of this family of proteins which can be estimated using the values of root mean square deviation (RMSD). The RMSD is the measure of the average distance between the backbone atoms of superimposed proteins. Very small RMSD values of 1.3 Å have been obtained when comparing the overall fold of Frequenin and Neurocalcin delta and of 1.7 Å between Frequenin and Recoverin. Similarly, the overall structure of Ca²⁺-bound non-myristoylated GCAP2 closely resembles that of Ca²⁺-bound non-myristoylated Recoverin (RMSD of 1.9 Å).⁶² Therefore, other members of the NCS family of proteins could potentially act as tags for purposes similar to those demonstrated here for Recoverin. RMSD>5 Å have been estimated for non-homologous proteins and ˜<2.5 Å for homologous proteins⁶³.

In accordance with a particular aspect of the invention there is provided the polynucleotide as defined herein, wherein the tag protein comprises a root mean square deviation (RMSD) of less than 2.5 Å, when compared to the 3D structure of Recoverin. More particularly, the invention provides the polynucleotide as defined herein, wherein the RMSD is less than 2 Å.

Thus, according to particular embodiments, the proteins GCAP1, GCAP2 et GCAP3, GCIP, KChIP1, KChIP2, Calsenilin/DREAM, NCS1/Frequenin, Neurocalcin delta, Hippocalcin, VILIP-1 maybe used as tags such as in the case of Recoverin.

### Tag

In accordance with a particular aspect of the invention, the Recoverin tag (TagR) cDNA can be cloned from freshly dissected bovine retina, although Recoverin from other organisms is deemed to be as useful. The RNA can be isolated and used for reverse transcription reaction. Then, first-strand cDNA can be used as a template for PCR using primers designed to amplify the coding sequence of Recoverin cDNA and, particularly to introduce restriction sites, more particularly NdeI and/or BamHI restriction sites.

In accordance with a particular aspect of the invention, the substantially full-length, or full-length Recoverin cDNA can be ligated into a plasmid to generate a Recoverin expression vector.

In accordance with a particular aspect of the invention, an EcoRI site in the native sequence of TagR can be changed to a silent mutation. More particularly, the EcoRI site (GAATTC) in the sequence of native Recoverin can be changed to GAGTTC (silent mutation) by site-directed mutagenesis.

Applicant has found that the presence of the myristoyl group did not improve the purification of TagR with its fusion partners. Therefore, in accordance with a particular aspect of the invention, the TagR is expressed without its myristoyl group.

In accordance with a particular aspect of the invention, the protein of interest is located at the C-terminus of the protein tag in order to allow the tag's major conformational change to occur at the N-terminus.

In accordance with a particular aspect of the invention, the TagR is located at the N-terminal of the protein of interest that is selected from, for example, but not limited to: tLRAT and RP2.

### Taq Engineering

This protein tag is further engineered to ease its use as a tag for recombination, expression, solubilisation and purification purposes.

In accordance with a particular aspect of the invention, the TagR has introduced in its nucleotide sequence one or more sequence encoding for a restriction site, such, as for example: NdeI and/or BamHI for ease of construction of the fusion cDNA sequence.

Alternatively, the polynucleotide comprises a silent mutation at GAATTC (e.g. within the coding sequence: mutation T to C, at position 330 from 5′) to avoid cleavage by the restriction enzyme specific to the EcoRI site during the construction of the cDNA.

In accordance with a particular aspect of the invention, the nucleotide sequence corresponding to a proteolytic cleavage site is introduced to the TagR nucleotide sequence, for ease of separation of the resulting protein from its fusion partner during purification.

Particularly, a nucleotide sequence encoding the specific thrombin cleavage site (LVPRGS) is inserted to the tagR sequence before the cDNA of the protein of interest. Alternatively, other proteolytic cleavage sites can be introduced such as, for examples, the TEV cleavage site corresponding to: ENLYFQ/G; or the HRV C3 cleavage site corresponding to LEVLFQ/GP.

In accordance with a particular aspect of the invention, the TagR protein is nonmyristoylated when expressed in prokaryotic expression systems, unless the system is complemented with enzyme and substrate for myristoylation⁴². Applicant has found that the presence of the myristoyl group did not improve the purification of TagR with its fusion partners. Therefore, in accordance with a particular aspect of the invention, the TagR can be expressed in basic prokaryotic expression systems without having to completement the system to allow myristoylation.

In accordance with a particular aspect of the invention, the TagR protein is complemented with a poly His tag positioned at the N-terminus of the protein of interest to show that TagR can be used in tandem with other tags.

In accordance with a particular aspect of the invention, the TagR protein is complemented with a further TagR positioned at the N-terminus of the first TagR to further enhance solubility of the resulting fusion protein. Such a procedure may be useful for the expression and purification of highly insoluble proteins.

### Nucleic Acid

In accordance with a particular aspect of the invention, the tag polypeptide encodes for Recoverin, particularly, the Recoverin has at least about 90% nucleic acid identity, more particularly at least 94% nucleic acid identity to SEQ ID NO: 1. More particularly, the Recoverin is substantially full length, or alternatively is about 23 kD.

In accordance with a particular aspect of the invention, the polynucleotide has introduced therein one or more sequence encoding for a restriction site, such, as for example: NdeI and/or BamHI. Alternatively, the polynucleotide comprises a silent mutation at GAATTC (e.g. EcoRI site within the coding sequence: mutation T to C at position 330 from 5′).

### Vectors and Expression Systems

In accordance with a further particular aspect, the invention provides for a vector comprising the polynucleotide as defined herein, operably linked to a promoter.

In accordance with a further aspect, the invention further provides an expression cassette comprising: a transcriptional initiation region functional in an expression host cell; the polynucleotide as defined herein; and a transcriptional termination region functional in the expression host cell.

Finally, in accordance with this particular aspect of the invention, there is provided a host cell comprising the expression cassette as defined herein above.

In accordance with a particular aspect of the invention, the substantially full-length, or full-length Recoverin cDNA can be ligated into a plasmid to generate a Recoverin expression vector. Particularly, the plasmid may be chosen from pET11a, pGEX-4T-3PEX Stiull, the plasmid may be chosen from the series of: pBAC, pAK, BJ, MP, pGPD, MW, pUCP, CY, MAT, pMSP, SNX, PM, etc. . . . well known in the art. More particularly, the plasmid may be chosen from several catalogs well known by the person skilled in the art such as from: addgene (www.addgene.org), thermofisher (www.thermofisher.com), millipore (www.emdmillipore.com), promega (www.promega.ca), EMBL (www.embl.de), GE Healthcare Life Sciences, Agilent, etc.

### Protein

In accordance with a further aspect, the invention further provides an isolated recombinant polypeptide encoded by the polynucleotide as defined herein, particularly, a recombinant protein comprising a tag encoded by the polynucleotide as defined herein.

Alternatively, the invention provides to a protein tag that has an amino acid sequence is at least about 40% identical, particularly at least 60% identical to SED ID No. 2.

Still, alternatively, the invention provides to a protein tag that has an amino acid sequence is at least about 90% identical, particularly at least 95% identical to SED ID No. 4. Even most particularly, the protein tag is as defined by SEQ ID NO. 4.

Also, alternatively, the tag protein lacks its native myristoyl group, which was found not to be essential for the purification of TagR with its fusion partners. Hence, in a particular embodiment, the polynucleotide is as defined in SEQ ID NO. 3.

### Method

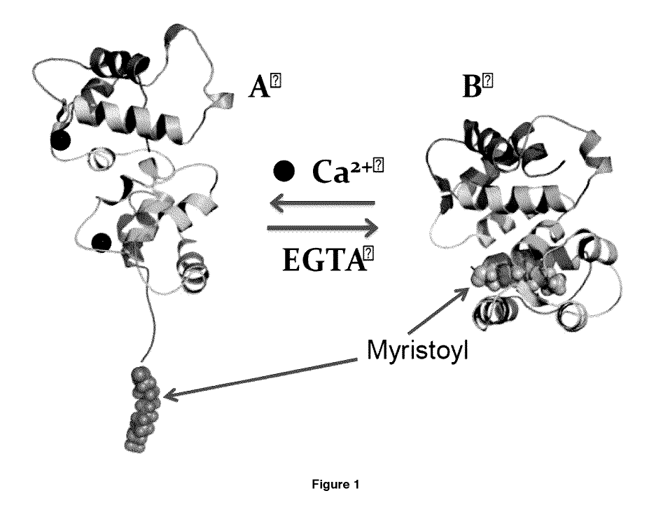

Recoverin's purification is based on its properties to reversibly bind calcium. Recoverin undergoes a large conformational change upon calcium binding²¹⁻²⁴ (FIG. 1). In the calcium-free state, the myristoyl group of Recoverin and several hydrophobic residues are sequestered in a deep hydrophobic box (FIG. 1B). However, calcium binding to two of its four EF-hands induces the extrusion of this myristoyl group as well as of several hydrophobic residues.²⁴ (FIG. 1A). The presence of calcium allows binding of TagR to the column as a result of the extrusion of its hydrophobic amino acids. However, of note, we have found that the presence of this myristoyl group did not improve the purification of TagR with its fusion partners.

Inversely, the chelation of the calcium ions by EGTA results in a large conformational change of TagR, that results in the sequestration of its hydrophobic residues inside the protein (FIG. 1B).

Therefore in accordance with a particular aspect of the invention, the purification of the protein of interest can be carried out in a single step hydrophobic chromatography, such as in the case for pure Recoverin²⁰. Applicant has found that the fusion of the protein of interest at the C-terminus of the TagR did not prevent the typical large conformational change of Recoverin taking place when calcium is removed using EGTA to allow its elution from the column.

Hence, the protein of interest can be cleaved and separated from the TagR by proteolysis, particularly using thrombin. The protein of interest is then separated from the TagR by simply adding calcium to the cleaved sample. As a result, Recoverin exposes its hydrophobic amino acids, thereby allowing its binding to the hydrophobic resin. This allows elution of the purified protein of interest free of its TagR fusion partner. TagR is then eluted separately using a buffer containing a calcium chelator such as, for example, EGTA, EDTA or BAPTA.

Therefore, in accordance with a particular aspect of the invention, there is provided a method for expressing, and purifying a protein of interest, comprising the steps of: expressing the protein fused to a substantially full-length TagR in an expression system; and purifying the fused-protein. Particularly, the purifying step is carried out by eluting the fused-protein from the hydrophobic affinity column in the presence of a calcium chelator; and optionally cleaving the eluted fused molecule in order to obtain the purified protein of interest cleaved from the TagR. Still, particularly, the purifying step is carried out by cleaving the fused-protein of interest from the TagR to obtain a cleaved protein/tag mixture, and then separating the cleaved protein of interest from the tag by eluting the cleaved protein of interest/tag mixture on a hydrophobic affinity column in the presence of calcium.

In accordance with a particular aspect, the invention also provides a method for producing a protein of interest, comprising growing a cell under conditions that permit expression of a protein of interest, wherein the cell comprises: a polypeptide as defined herein; or a vector as defined herein, or an expression cassette as defined herein.

According to a particular embodiment, a significantly higher level of expression and of solubility is obtained when the cell transfected with the fused-polynucleotide of the invention is cultured in the minimal growth medium than in the normal LB growth medium (all culture conditions are the same except for the growth medium). Indeed, some insoluble proteins are more strongly expressed and more soluble in minimal growth medium. Therefore, In accordance with a particular aspect, the invention also provides a method for producing a protein of interest, particularly an insoluble protein, the method comprising growing a cell as defined herein under minimal growth conditions to allow better folding of the insoluble protein.

### Presence of Detergent

TagR also allows purification of highly insoluble proteins in the presence of detergent, which cannot be achieved using GST. Indeed, SDS does not prevent fused TagR-protein binding to the column used for purification.

### Kits

In accordance with a particular aspect, the invention also provides a kit for the expression and purification of a protein of interest, the kit comprising: a polynucleotide according as defined herein; and/or instructions on how to insert the protein in a suitable vector; and/or instructions on how to transform the vector in a host cell; and/or instructions on how to isolate a recombinant fused-protein from the host cell; and/or instructions on how to purify the recombinant fused-protein.

Alternatively, the kit for the expression and purification of a protein of interest comprises: a vector as defined herein, or an expression cassette as defined herein; and/or instructions on how to transform the vector in a host cell; and/or instructions on how to isolate a recombinant fused-protein from the host cell; and/or instructions on how to purify the recombinant fused-protein according to the method of the invention.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The properties of Recoverin to improve expression and solubility of fusion protein partners have thus been compared with those of the GST tag in the following examples.

### EXAMPLES

### Example 1—Materials and Methods

1.1 Cloning of tLRAT, RP2 and Recoverin

Two proteins of interest have been used in the present study: truncated lecithin retinol acyltransferase (tLRAT, amino acids 31-196) and retinitis pigmentosa 2 (RP2). Human tLRAT has been cloned in the plasmid pET11a (Novagen) as previously described.^{47,48} Briefly, RNA from freshly dissected human retinal pigment epithelium was isolated with Tri-reagent (Sigma) and used for reverse transcription reaction with the RevertAid® H minus First Strand cDNA Synthesis Kit (Fermentas). In order to insure unidirectional cloning, additional NdeI and Bpu1102 I restriction sites were attached to the 5′ end of each primer. The pET11a vector was then linearized with NdeI and Bpu1102 I and subsequently ligated with the purified PCR product corresponding to tLRAT.

The human RP2 construct cloned in the pGEX-4T-3 plasmid to express a GST fusion protein was a kind gift from Dr. Alfred Wittinghofer (Max-Planck-Institut für Molekulare Physiologie, Germany). Recoverin (TagR) cDNA has been cloned from freshly dissected bovine retina to produce the pET11a-Rec vector as previously described.⁴² RNA was isolated with Tri-reagent and used for reverse transcription reaction (RevertAid Kit). Then, first-strand cDNA was used as a template for PCR using primers designed to amplify the coding sequence of Recoverin cDNA and to introduce NdeI and BamHI restriction sites. The full-length bovine cDNA of Recoverin was then ligated into the pET11a plasmid.

1.2 Preparation of the Different Constructs of tLRAT and RP2 in Fusion with TagR or GST in pET11a and pGEX-4T-3 or in Tandem with the PolyHis Tag

The coding region of tLRAT or RP2 has been inserted between the BamHI and EcoRI sites of the pET11a-Rec vector (with no stop codon). It is noteworthy that there was an EcoRI site (GAATTC) in the sequence of TagR which has been changed to GAGTTC (silent mutation) by site-directed mutagenesis (QuikChange Lightning, Agilent). Moreover, 5 glycines and then a specific thrombin cleavage site (LVPRGS) were inserted before the cDNA of tLRAT or RP2. The final constructions are as follows: BamHI-TagR-5 glycines-Thrombin cleavage site-tLRAT or RP2-EcoRI. RP2 was provided in fusion with GST (pGEX-4T-3, see above). It included 5 glycines and the thrombin cleavage site, such as described above for pET11a. The tLRAT coding sequence has been inserted between the BamHI-EcoRI sites of the pGEX-4T-3 vector. The TagR and GST tags were thus located at the N-terminal of tLRAT and RP2. The polyHis (10 histidines) tag was also used in tandem with TagR using the TagR-tLRAT construct described above. Thus, 10 histidines and 5 glycines have been added at the N-terminal of TagR by PCR using appropriate primers to produce the PolyHis-TagR-tLRAT construct.

### 1.3 Expression of TagR-RP2, TagR-tLRAT, GST-RP2 and GST-tLRAT

Plasmid DNA of TagR-tLRAT, TagR-RP2, GST-tLRAT and GST-RP2 were transformed into E. coli BI21(DE3) RIPL (Novagen) and grown overnight in the LB medium until saturation. Then, fresh LB containing 50 μg/ml ampicillin and chloramphenicol was inoculated with the transformed culture and incubated at 37° C. under agitation (250 rpm) until A_{600 nm}=0.6. Their expression was then induced with isopropyl β-D-thiogalacto-pyranoside (IPTG) (0.5 and 0.1 mM for the constructions in the pET11a or the pGEX vectors, respectively) followed by an incubation for 16 h at 21° C. Bacteria were then sedimented by centrifugation at 3,275 g for 25 min. Bacteria lysis was done first using 250 μl of lysozyme at a concentration of 4 mg/ml, which includes 1 mM PMSF (phenylmethylsulfonyl fluoride, a protease inhibitor). The resuspended cells were kept on ice for 30 min. The cells were then sonicated and centrifuged at 20,000 g for 30 min at 4° C. It is noteworthy that TagR was expressed without its myristoyl group⁴² (FIG. 1) as we have found that the presence of this group did not improve the purification of TagR with its fusion partners. Some experiments have been carried out by expressing fusion proteins also for 16 h at 21° C. in the minimal growth medium containing NaH₂PO₄, Na₂HPO₄, KH₂PO₄, K₂HPO₄, betaine, Na₂SO₄, NH₄Cl, 0.2% glucose, thiamine and MgSO₄ and the salts of Studier⁴⁹ (for comparison, the LB medium contains a much larger amount of nutrients: tryptone and yeast extract and NaCl).

### 1.4 Purification of TagR-RP2 and TagR-tLRAT

The pellet from 50 ml bacterial culture resulting from the expression of TagR-RP2 or TagR-tLRAT have respectively been resuspended in 4.75 ml of buffer A (50 mM Hepes (pH 7.5), 100 mM NaCl, 1 mM CaCl₂, 5 mM β-mercaptoethanol) or of buffer B (5 mM Hepes (pH 7.5), 1 mM CaCl₂, 5 mM β-mercaptoethanol). After bacteria lysis (see Example 1.3) and centrifugation (20,000 g for 30 min at 4° C.), the cleared lysate was loaded to a column containing 5 ml of phenyl Sepharose 6 Fast Flow (low sub resin; GE Healthcare) that had been preequilibrated with buffer A (TagR-RP2) or B (TagR-tLRAT). The purification of these fusion proteins was performed at 4° C. The presence of 1 mM calcium allows binding of TagR to the column as a result of the extrusion of its hydrophobic amino acids (FIG. 1A). The column was then washed with at least 10 column volumes of buffer A (TagR-RP2) or B (TagR-tLRAT) to remove nonspecifically adsorbed proteins. Then, the fusion protein was eluted with buffer C containing 5 mM Hepes at pH 7.5, 100 mM NaCl, 5 mM β-mercaptoethanol and 1 mM EGTA. Indeed, the chelation of the calcium ions by EGTA leads to a large conformational change of TagR which results in the sequestration of its hydrophobic residues inside the protein (FIG. 1B). The cleavage of TagR from its fusion partner was then achieved in solution using thrombin (GE Healthcare). One cleavage unit digests 100 μg of fusion protein at 4° C. for 48 hours with gentle agitation. Then, 10 mM CaCl₂ was added to the cleaved fusion proteins and loaded on the same column that had been preequilibrated with the buffer A (TagR-RP2) or B (TagR-tLRAT) containing 10 mM CaCl₂. Purified RP2 does not bind to the column and is collected in the first fractions. Thrombin was removed from the eluent by connecting a Hitrap Benzamidine FF sepharose column (GE Healthcare) to the phenyl Sepharose column; benzamidin is a synthetic inhibitor of thrombin covalently coupled to the resin. TagR and the eventually remaining uncleaved TagR-RP2 fusion protein bind to the column containing 10 mM CaCl₂. TagR and the fusion protein were eluted with buffer D containing 5 mM Hepes, 100 mM NaCl, 15 mM EGTA, 5 mM β-mercaptoethanol. In contrast, cleaved tLRAT strongly binds to the hydrophobic column by virtue of its high hydrophobicity (see Example 5.1). After washing the column with buffer D, which allows eliminating thrombin and eluting TagR and the eventually remaining uncleaved TagR-tLRAT fusion protein, pure tLRAT was eluted with pure water. Concentrated SDS (sodium dodecyl sulfate) and citrate buffer was then immediately added to purified tLRAT to reach a final concentration of 0.05% and 10 mM, respectively, to avoid its precipitation.

### 1.5 Binding of TagR-tLRAT to the Hydrophobic Column in the Presence of SDS and Purification of the Fusion Protein

The TagR-tLRAT fusion protein has been expressed as described in Example 1.3 except that bacterial culture has been resuspended in buffer A (Example 1.4) containing also 0.05% SDS, which allowed extensive solubilisation of the fusion protein. The supernatant of the centrifugation has been loaded on the phenyl Sepharose 6 Fast Flow column. The largest share of the protein binds to the column in the presence of 0.05% SDS, which is not true for the GST tag. The column was then washed with at least 20 column volumes of the same buffer without SDS (cleavage of the fusion protein cannot be achieved with thrombin in the presence of SDS). TagR-tLRAT was eluted with buffer C (see Example 1.4). Cleavage of TagR-tLRAT has not been assayed in these particular experiments but it can be either performed directly on the column, such as described in Example 1.6, or after the elution of the fusion protein. Purified TagR-tLRAT remains soluble in the absence of SDS.

1.6 Expression, Purification of PolyHis-TagR-tLRAT, Cleavage of PolyHis-TagR and Purification of tLRAT

The PolyHis-TagR-tLRAT fusion protein has been expressed as described for TagR-tLRAT (see Example 1.3). The pellet from 50 ml of the bacterial culture is resuspended in 10 ml of a lysis buffer (100 mM Tris, pH 7.8, 100 mM NaCl, 1 mM EDTA, 1 mM EGTA, 1.4 μg/μl aprotinin). Bacteria were first disrupted by 3 cycles of freeze-thawing and the cell suspension was then sonicated during 3 min (cycles of 5 s) on ice. Bacteria were then centrifuged at 13,000×g during 30 min. Supernatant was discarded and the membrane pellet was resuspended in the loading buffer (500 mM Tris, 5 mM imidazole, 0.1% SDS, pH 7.8). The supernatant was then loaded on a His-Trap column (GE Healthcare). The column was sequentially washed with 200 ml of a buffer containing 500 mM Tris (pH 7.8), 30 mM imidazole and 1 mM CaCl₂ and then with an additional buffer containing 50 mM Tris (pH 7.8), 100 mM NaCl, 0.1 M NDSB201 (3-(1-pyridinio)-1-propanesulfonate, Sigma-Aldrich) and 1 mM CaCl₂. The PolyHis-TagR has then been cleaved from tLRAT using thrombin directly on the column for 40 hours at room temperature. Furthermore, the column was washed with 6 column volumes with the buffer used for the cleavage (50 mM Tris, pH 7.8, 100 mM NaCl, 0.1 M NDSB201, 1 mM CaCl₂), which allowed to remove thrombin. tLRAT has then been eluted using a buffer containing 50 mM Tris, pH 7.8, 100 mM NaCl, 1 mM CaCl₂, 0.1% SDS and 1 mM DTT. Finally, the cleaved PolyHis-TagR and the uncleaved fusion protein have been eluted with a buffer including 500 mM Tris, pH 7.8, 150 mM imidazole and 0.1% SDS.

### 1.7 Polyacrylamide Gel Electrophoresis and Western Blot

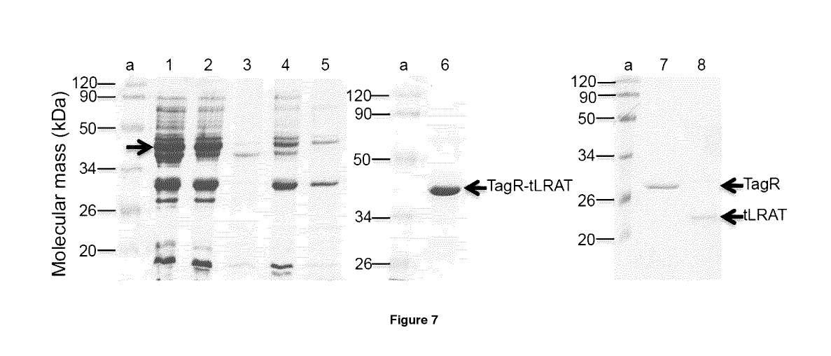

The analysis of the level of expression and purity of the proteins was carried out on a Bio-Rad Mini-protean II electrophoresis cell. The protein samples were separated using 15% acrylamide SDS-PAGE (SDS-polyacylamide gel electrophoresis) (or 12% acrylamide, see Example 4.2 and FIG. 7). The electrophoresis gels were then Coomassie stained and protein identification was performed using a prestained protein molecular mass marker (Thermo Fisher Scientific). The identity of the bands attributed to TagR-tLRAT, GST-tLRAT, TagR-RP2 and GST-RP2 as well as purified tLRAT and RP2 has been confirmed by western blot using antibodies against these specific proteins as well as by mass spectrometry for purified tLRAT, RP2 and Recoverin.

### Example 2—Comparison Between the Dependence of the Protein Expression Level on the Type of Vector and of Fusion Taq

2.1 Comparison Between Protein Expression in Fusion with TagR Using Different Vectors

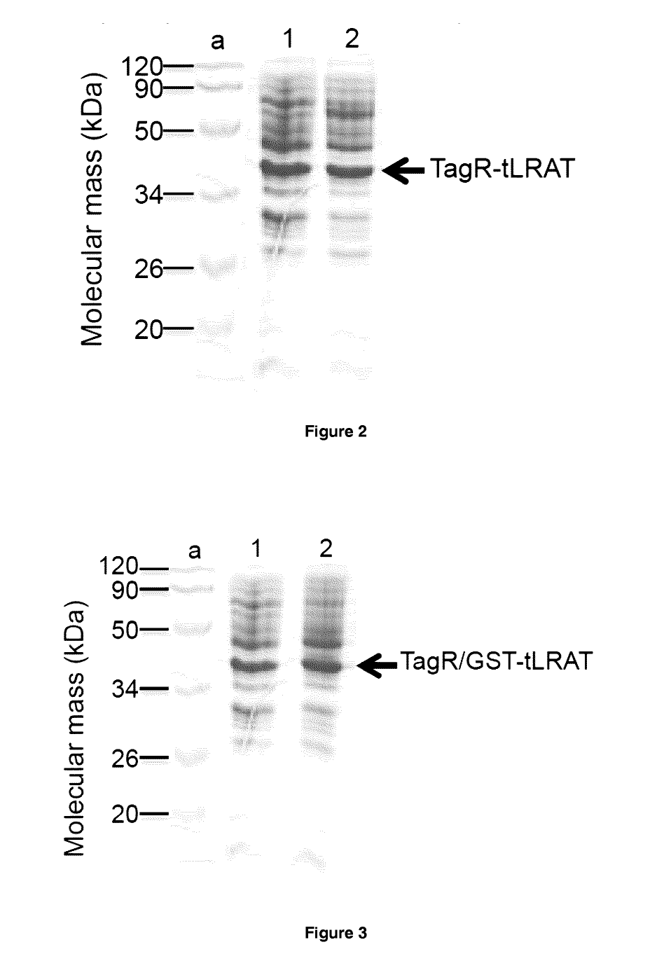

Fusion tags can potentially increase the total yield of proteins of interest by improving their expression and decreasing their degradation.² However, the level of protein expression is highly dependent on the promoter present in the cloning vector. Expression of TagR-tLRAT in the pET11a and pGEX-4T-3 expression vectors has thus been performed using the same culture conditions. Their level of expression has then been compared by SDS-PAGE after bacteria lysis. As shown in FIG. 2, a similar level of expression has been obtained with TagR-tLRAT when cloned in pET11a or pGEX-4T-3 (see arrow). Indeed, the intensity of the band of TagR-tLRAT is similar when expressed using these two vectors. The same experiments have been carried out with TagR-RP2 with similar results. Therefore, pET11a and pGEX-4T-3 can be equally used to express proteins of interest in our experiments.

2.2 Comparison Between Protein Expression in Fusion with TagR and GST Using the Same Vector

tLRAT has been expressed in fusion with either TagR or GST in the vector pGEX-4T-3 using the same conditions. As can be seen in the SDS-PAGE of the lysed bacteria shown in FIG. 3, the level of expression of tLRAT in fusion with TagR is similar to that in fusion with GST. Indeed, the intensity of the band of TagR-tLRAT is similar to that corresponding to GST-tLRAT (see arrow). Therefore, there is no significant difference between the expression level of these two proteins of interest in fusion with TagR or GST. In this context, it can thus be concluded that TagR has properties similar to GST with regards to its ability to improve expression of these two proteins.

### Example 3—Comparison Between the Solubility of Fusion Proteins Using TagR and GST Tags

3.1 RP2 Solubility is Much Larger in Fusion with TagR than with GST

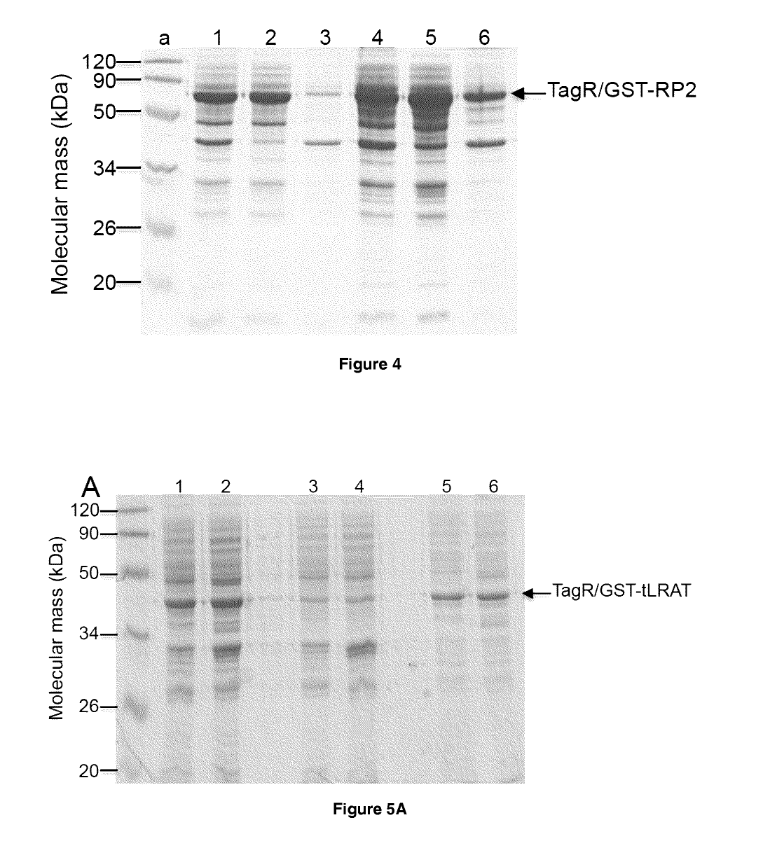

A similar level of expression of the same fusion protein has been obtained with the pET11a or pGEX-4T-3 vectors (FIG. 2) as well as of the same protein of interest in fusion with TagR or GST in the same vector (FIG. 3). TagR-RP2 in pET11a and GST-RP2 in pGEX-4T-3 have thus been expressed using the same conditions to compare their expression level. FIG. 4 is showing that a larger level of expression is obtained for GST-RP2 (lane 5) than for TagR-RP2 (lane 1) after lysis of the bacteria (see the arrow for the bands corresponding to TagR-RP2 and GST-RP2). TagR-RP2 is however more soluble than GST-RP2. Indeed, the centrifugation of the lysed bacteria allowed the analysis of the supernatant (soluble fractions, lanes 2 and 4) and of the pellet (insoluble fractions, lanes 3 and 6). It can be seen that the amount of insoluble TagR-RP2 (lane 3) is very small compared to that of GST-RP2 (lane 6) in the pellet. Accordingly, the ratio between the amount of soluble (lane 2) and insoluble (lane 3) TagR-RP2 is much larger than with GST-RP2 (compare lanes 4 and 6). The TagR thus provides a much better solubility for RP2 than in the case of GST.

3.2 Comparison Between tLRAT Solubility in Fusion with TagR or GST and in Different Growth Media

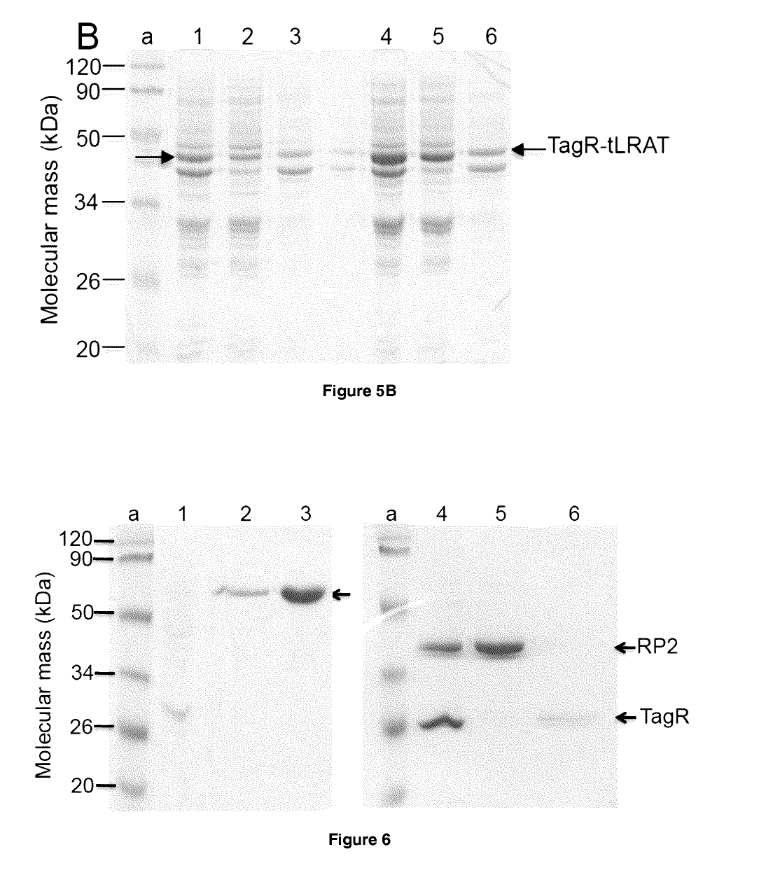

A similar solubility has been obtained with TagR-tLRAT or GST-tLRAT in pGEX-4T-3. Indeed, as can be seen in FIG. 5A, the amount of soluble TagR-tLRAT (lane 3) in the supernatant is similar to that of GST-tLRAT (lane 4). Accordingly, similar amounts of insoluble TagR-tLRAT (lane 5) and GST-tLRAT (lane 6) have been obtained. FIG. 5A also shows that the a similar level of expression of tLRAT has been obtained in fusion with TagR-tLRAT (lane 1) or GST-tLRAT (lane 2) after bacteria lysis, which is also consistent with the data shown in FIG. 3. The TagR and GST thus provide a similar, albeit low, solubility for tLRAT. These data thus suggest that the TagR and GST tags can be equally used to solubilize tLRAT but that these fusion partners are not very efficient to solubilize tLRAT. This is consistent with the high hydrophobicity of this protein (see Example 5.1). However, TagR performs better than GST when proteins are expressed using a minimal growth medium (FIG. 5B).

As can be seen in FIG. 5B, a significantly larger level of expression and of solubility is obtained when TagR-tLRAT in pGEX-4T-3 is cultured in the minimal growth medium than in the normal LB growth medium (all culture conditions are the same except for the growth medium). Indeed, the intensity of the band of TagR-tLRAT in the minimal growth medium (lane 4) is much higher than that in the normal LB growth medium (lane 1). TagR-tLRAT is thus more strongly expressed in the minimal growth medium. In addition, the solubility of TagR-tLRAT is much higher when grown in the minimal growth medium (lane 5) than that in the normal LB growth medium (lane 2). A higher amount of insoluble TagR-tLRAT is produced using the normal LB growth medium (lane 3) than the minimal growth medium (lane 6) when compared to the respective amount of soluble proteins in lanes 2 and 5. Full solubilisation of tLRAT requires the presence of SDS (see Example 5.1). In contrast, no expression was obtained for GST-tLRAT in the minimal growth medium. Therefore, the expression and solubility of GST-tLRAT using in the LB and the minimal growth media cannot be compared and these results are thus not shown.

### Example 4—Purification of TagR Fusion Proteins, Cleavage of TagR and Purification of the Proteins of Interest

### 4.1 Purification of TagR-RP2, Cleavage of TagR and Purification of RP2

FIG. 6 shows that highly purified TagR-RP2 has been obtained using a single step hydrophobic chromatography on phenyl Sepharose. Highly purified TagR-RP2 has been eluted using EGTA (lane 3, see arrow), such as in the case of pure Recoverin.⁴² These data thus suggest that the fusion of RP2 with Recoverin did not prevent the large conformational change of Recoverin taking place when calcium is removed using EGTA (FIG. 1B) to allow its elution from the column. Moreover, quantitative cleavage is obtained upon proteolysis using thrombin as no fusion protein remains after protein cleavage (lane 4). Pure RP2 has then been separated from TagR by adding calcium to the cleaved sample. As a result, TagR exposes its hydrophobic amino acids (FIG. 1A), thereby allowing its binding to the phenyl Sepharose resin. This allowed elution of purified RP2 free of its TagR fusion partner (lane 5, see arrow). TagR is then eluted using a buffer containing EGTA (lane 6, see arrow). Therefore, RP2 can be highly expressed in fusion with TagR, the TagR fusion partner can be successfully cleaved and purified RP2 is obtained free of contaminants. In this regard, TagR (present data) and GST⁵⁰ perform similarly to express and purify RP2. However, TagR allows cleavage both on the column and in solution whereas cleavage must be performed on the column in the case of GST to avoid dialysis of the bound glutathione.

4.2 Purification of TagR-tLRAT, Cleavage of TagR and Purification of tLRAT

FIG. 7 shows that highly purified TagR-tLRAT has been obtained using a single step hydrophobic chromatography on phenyl Sepharose as well as that cleavage of this fusion protein and purification of tLRAT can be achieved. As shown in FIG. 5B, a large share of TagR-tLRAT is soluble when expressed in bacteria cultured in a minimal growth medium. It is noteworthy that TagR-tLRAT requires the presence of SDS to be fully solubilised from the bacteria lysate (see Example 5.1). It can nevertheless be seen in FIG. 7 that the intensity of the band of TagR-tLRAT in the total lysate (lane 1, see arrow) is similar to that of TagR-tLRAT in the supernatant (lane 2) after centrifugation of the total lysate whereas very little TagR-tLRAT, if any, can be found in the pellet (lane 3). TagR-tLRAT is thus highly soluble. Highly purified TagR-tLRAT has been eluted using EGTA (lane 6, see arrow), such as in the case of pure TagR-RP2 (see Example 4.1), after an extensive washing of the column (lane 5). Moreover, TagR-tLRAT has then been cleaved using thrombin (data not shown). TagR was eluted using a buffer containing EGTA (lane 7, see arrow) whereas purified tLRAT free of its TagR fusion partner (lane 8, see arrow) was eluted using pure water likely because it is a very highly hydrophobic protein, which likely results in a strong binding of tLRAT to the hydrophobic column. It is noteworthy that tLRAT cannot be eluted using a buffer containing SDS. It must be stressed that SDS must be immediately added to the purified tLRAT fractions to avoid protein precipitation.

### Example 5—Demonstration that the TaqR Fusion Taq can Allow Protein Purification Alone or in Tandem with an Another Taq in the Presence of SDS

### 5.1 Binding of TagR-tLRAT to the Hydrophobic Column in the Presence of SDS and Purification of the Fusion Protein

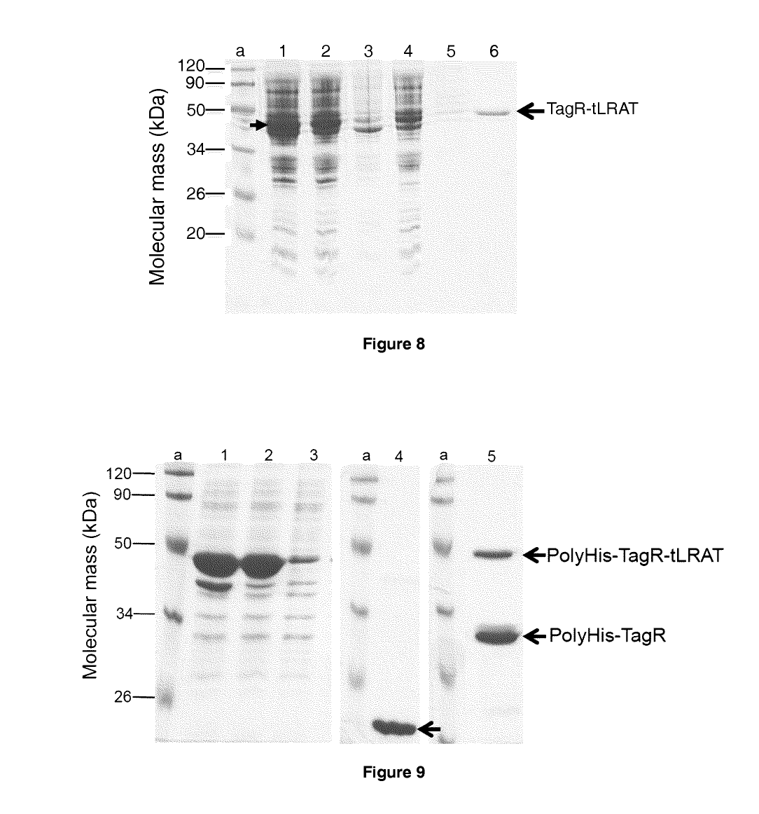

tLRAT is a very hydrophobic protein and it requires the presence of detergent for its solubilisation in the absence of a highly soluble fusion partner. We have indeed shown that, among the large number of detergents assayed, only SDS (0.05% p/v; cmc ˜0.17-0.23%) allowed full solubilisation of tLRAT.⁴⁸ Large concentrations of SDS could result in protein denaturation.⁵¹ We have thus demonstrated that tLRAT enzymatic activity remains unchanged from 0.05% up to 1% SDS and that this activity is 55,000 times higher than the highest activity reported in the literature.⁴⁸ It can therefore be concluded that this range of concentrations of SDS has no detrimental effect on tLRAT. As shown in FIG. 8, adding 0.05% SDS to the bacteria lysate allowed solubilisation of the largest share of TagR-tLRAT (lane 1). TagR-tLRAT can be almost solely found in the supernatant after centrifugation (compare the intensity of the band of TagR-tLRAT in the soluble and insoluble fractions in lanes 2 and 3, see arrow, respectively). The pellet indeed contains little fusion protein (lane 3). FIG. 8 demonstrates that a large share of TagR-tLRAT binds to the phenyl Sepharose column in the presence SDS (lane 4) whereas no binding of GST to the glutathione column is observed in the presence of SDS.⁴¹ The column has been extensively washed (lane 5) to remove any trace of SDS to allow TagR-tLRAT cleavage on the column using thrombin (not assayed in the present experiments). Finally, TagR-tLRAT has been eluted using the EGTA buffer in the absence of SDS (lane 6).

5.2 Purification of PolyHis-TagR-tLRAT, Cleavage of PolyHis-TagR and Purification of tLRAT

Dual tags, or tandem purification procedures, have been developed in recent years.^{1,4,9,11,12,22,23} It consists of two different tags that can either be cloned both at the N- or C-terminal or one at N-terminal and the other one at the C-terminal. These tags can however be more easily cleaved if they are located at a single end of the protein of interest. Dual tags should preferably include a solubilisation as well as a purification tag. A single purification step can be achieved if the cloning strategy is properly designed but this has hardly been achieved until now. In our experiments, the PolyHis tag has been cloned at the N-terminal of TagR in fusion with tLRAT to produce the PolyHis-TagR-tLRAT construction. Purification has been achieved using the immobilized metal affinity chromatography which allowed binding of the PolyHis tag in the presence of SDS. Purification could also likely be performed using TagR properties to bind the hydrophobic phenyl sepharose resin in the presence of SDS as shown in FIG. 8. SDS allows an extensive solubilisation of the PolyHis-TagR-tLRAT fusion protein (compare lanes 1 and 2, FIG. 9). After an extensive washing of the column, thrombin was injected into the column and incubated for 36 h. It allowed cleavage tLRAT from the PolyHis-TagR tag. Pure tLRAT was then eluted from the column using SDS (lane 4). The uncleaved fusion protein and cleaved PolyHis-TagR were finally eluted using imidazole (lane 5).

### Example 6—Dual-tagsR Tagged Protein

A dual TagsR is used to further enhance solubility of a protein of interest with no compromise on its properties to bind the hydrophobic phenyl Sepharose resin. For this purpose, a second Recoverin molecule is cloned at the C-terminal of a first Recoverin fusion partner. The expression and purification are carried out as already described.

### Discussion

When comparing the properties of TagR and GST, we observed that the pellet of bacteria transformed with pGEX-4T-3 was typically much larger than that obtained pET11a. This however hardly resulted in the expression of a higher amount of proteins of interest, such as observed in FIG. 2. However, a higher expression of GST-RP2 in pGEX-4T-3 than of TagR-RP2 in pET11a was observed which, however, resulted in a lower proportion of soluble GST-RP2 than of TagR-RP2 (FIG. 4).

Although increasing protein expression is an interesting outcome, the main challenge is to get a soluble protein. In fact, high protein expression may be detrimental for protein solubility. Indeed, at high expression rates, protein folding may not be as efficient as when performed at lower expression rates. This can be well appreciated in FIG. 5B where production of soluble protein was compared when bacteria were cultured in normal LB and minimal expression growth media. The data clearly show that more soluble, likely better folded, proteins are produced when protein production is slowed down by virtue of the minimal expression growth media which contains a small amount of nutrients.¹⁴

We have purposely chosen to perform a large share of the experiments using tLRAT, a protein which is difficult to solubilize. In fact, tLRAT is little soluble with either the GST or TagR fusion partners as shown in FIG. 5A. However, as mentioned above, TagR-tLRAT becomes much more soluble using bacteria cultured in minimal growth medium (FIG. 5B) whereas no protein expression of GST-tLRAT can be obtained in these conditions. This is thus a clear advantage of the TagR technology compared to GST. TagR also allows tLRAT purification in the presence of detergent which cannot be achieved using GST. Indeed, SDS does not prevent TagR-tLRAT binding to the column used for its purification (FIG. 8) whereas no binding of GST fusion proteins to the glutathione resin can be observed in the presence of the same detergent.⁴¹ A major concern from the industry is that the technology should allow solubilisation of a large range of proteins of interest from lower to higher molecular mass proteins.

We predict that dual TagRs (one TagR cloned at the C-terminal of another TagR) fusion partners would allow to further enhance solubility of proteins of interest with no compromise on its properties to bind the hydrophobic phenyl Sepharose resin. Dual TagRs would then represent an improved technology with regards to the solubilisation of «difficult-to-solubilize» proteins of interest. Most important would be that purification of proteins of interest can be achieved even though a detergent must be used for their solubilisation, which was shown to be possible with TagR as a fusion partner (FIG. 8).

An additional concern from the industry is the production of a soluble protein of interest after its cleavage from its fusion partner. RP2 and tLRAT were shown to remain soluble after their cleavage from TagR (FIGS. 6 and 7). SDS allows to prevent precipitation of tLRAT (FIGS. 7 and 9). Therefore, we have succeeded to purify a «difficult-to-solubilize» protein of interest (tLRAT) which remained soluble after its cleavage and purification, thereby satisfying the interests of the industry.

Dual tagging methods have been recently used by combining, for example, a solubility-enhancing tag and a purification tag. This procedure can however complicate the removal of the tags unless the construction is carefully planned. We have thus added a PolyHis tag at the N-terminal of TagR to find out if they could be used in tandem. This approach allowed to purify tLRAT and to remove the PolyHis-TagR tags at the same time (FIG. 9) such as when TagR alone was used (FIG. 7).

GST is by far the most widely used protein tag because of its high solubility and also since it allows protein purification typically using a single step affinity chromatography using a resin that is covalently coupled with its glutathione substrate. However, the use of detergents prevents binding of the GST tag to the glutathione resin,⁴¹ which thus limits its use for a large range of proteins, notably those associated with membranes as they typically require detergent for their solubilisation. In addition, the high affinity of GST for glutathione prevents the direct purification the protein of interest from cleaved GST after protease digestion. Indeed, dialysis must first be performed to remove glutathione from GST, which lengthens the purification procedure and typically leads to a decrease of protein yield. In contrast, the presence of detergents does not prevent protein purification of the TagR fusion proteins assayed. Moreover, concerns have been raised with regard to the slow kinetic of binding of GST to its resin which results in a highly time-consuming loading of the cell lysates to the chromatography column.⁵²

Maltose binding protein (MBP)⁵³ is the second mostly used protein tag. It has been shown to increase the solubility of a number of proteins of interest.^{54,55} It was also found to be attractive because it allows affinity purification with a low cost resin covalently coupled with the amylose substrate of MBP. However, we and others⁵⁶⁻⁵⁸ have found that MBP fusion proteins fail to properly bind to the amylose resin. Consequently, MBP is often used in combination with other tags to achieve purification of its fusion partner which, however, lengthens the purification procedure and leads to a decrease of protein yield and an increase of the cost of these experiments.² Therefore, although it is an inexpensive technology, the difficulty to achieve purification of its fusion protein partner in a single step using affinity chromatography has led to a decrease in the interest for this protein tag.

Thioredoxin⁵⁹ and ubiquitin-like modifier (SUMO)⁶⁰ have attracted less interest, even though they enhance solubility, because they are not purification tags. Consequently, they must be used in combination with small purification tags to achieve protein purification, which complicates the procedure. It is also noteworthy that the SUMO technology is very expensive. These protein tags are thus less appealing.

As shown herein, the TagR technology allows a more efficient expression and solubilisation of its fusion partner than GST. Moreover, it allows purification and cleavage of the fusion partner. In addition, it is cheaper to use than GST and the very popular small PolyHis tag. Indeed, the commercially available resins used to purify GST, PolyHis and TagR respectively costs 62$, 43$ and 34$ to purify 10 mg of protein. The TagR thus appears as a new, attractive technology to improve the expression and solubility of proteins of interest. Moreover, it allows purification of its fusion partner and it can be easily removed by proteolytic cleavage.

### CONCLUSION

We are presenting herein, a new protein tag to improve the solubility and purification of proteins of interest. This new protein tag has been called TagR. It is the widely studied 23 kDa protein Recoverin, which possesses properties typical of protein tags. Indeed, Recoverin is highly soluble (>30 mg/ml), it can be highly purified in a single step and its expression level in bacteria is very high (>30 mg/L of culture).⁴² Its purification is based on its properties to reversibly bind calcium through a calcium-myristoyl switch. Recoverin undergoes a large conformational change upon calcium binding.⁴³⁻⁴⁶ Indeed, calcium binding to two of its four EF-hands induces the extrusion of its myristoyl group as well as of several hydrophobic residues (FIG. 1A).⁴⁶ In the calcium-free state, the myristoyl group of Recoverin and several hydrophobic residues become sequestered in a deep hydrophobic pocket (FIG. 1B). Purification is thus performed using hydrophobic chromatography where the calcium-bound Recoverin strongly binds to the resin whereas the calcium-free protein can be eluted free of contaminants.⁴²

We have previously shown that the presence of the myristoyl group is not necessary to achieve high purity of Recoverin.⁴² The properties of TagR to improve solubility of fusion protein partners and to allow their purification have thus been compared with those of the same proteins of interest using the GST tag.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

All patents, patent applications and publications mentioned in this specification are herein incorporated by reference to the same extent as if each independent patent, patent application, or publication was specifically and individually indicated to be incorporated by reference.

### REFERENCES

[1] Arnau, J, Lauritzen, C, Petersen, G E, and Pedersen, J. (2006) Current strategies for the use of affinity tags and tag removal for the purification of recombinant proteins, Protein Expr Purif 48, 1-13.
[2] Bell, M R, Engleka, M J, Malik, A, and Strickler, J E. (2013) To fuse or not to fuse: what is your purpose?, Protein Sci 22, 1466-1477.
[3] Chen, R. (2012) Bacterial expression systems for recombinant protein production: E. coli and beyond, Biotechnol Adv 30, 1102-1107.
[4] Costa, S, Almeida, A, Castro, A, and Domingues, L. (2014) Fusion tags for protein solubility, purification and immunogenicity in: the novel Fh8 system, Front Microbiol 5, 63.
[5] Esposito, D, and Chatterjee, D K. (2006) Enhancement of soluble protein expression through the use of fusion tags, Curr Opin Biotechnol 17, 353-358.
[6] Lichty, J J, Malecki, J L, Agnew, H D, Michelson-Horowitz, D J, and Tan, S. (2005) Comparison of affinity tags for protein purification, Protein Expr Purif 41, 98-105.
[7] Malhotra, A. (2009) Tagging for protein expression, Methods Enzymol 463, 239-258.
[8] Papaneophytou, C P, and Kontopidis, G. (2014) Statistical approaches to maximize recombinant protein expression in Escherichia coli: a general review, Protein Expr Purif 94, 22-32.
[9] Pina, A S, Lowe, C R, and Roque, A C. (2014) Challenges and opportunities in the purification of recombinant tagged proteins, Biotechnol Adv 32, 366-381.
[10] Terpe, K. (2003) Overview of tag protein fusions: from molecular and biochemical fundamentals to commercial systems, Appl Microbiol Biotechnol 60, 523-533.
[11] Waugh, D S. (2005) Making the most of affinity tags, Trends Biotechnol 23, 316-320.
[12] Young, C L, Britton, Z T, and Robinson, A S. (2012) Recombinant protein expression and purification: a comprehensive review of affinity tags and microbial applications, Biotechnol J 7, 620-634.
[13] Zhao, X, Li, G, and Liang, S. (2013) Several Affinity Tags Commonly Used in Chromatographic Purification, J Anal Methods Chem 2013, 581093.
[14] Almo, S C, and Love, J D. (2014) Better and faster: improvements and optimization for mammalian recombinant protein production, Curr Opin Struct Biol 26, 39-43.
[15] Xiao, S, Shiloach, J, and Betenbaugh, M J. (2014) Engineering cells to improve protein expression, Curr Opin Struct Biol 26, 32-38.
[16] Hannig, G, and Makrides, S C. (1998) Strategies for optimizing heterologous protein expression in Escherichia coli, Trends Biotechnol 16, 54-60.
[17] Demain, A L, and Vaishnav, P. (2009) Production of recombinant proteins by microbes and higher organisms, Biotechnol Adv 27, 297-306.
[18] Bandaranayake, A D, and Almo, S C. (2014) Recent advances in mammalian protein production, FEBS Lett 588, 253-260.
[19] Amarasinghe, C, and Jin, J P. (2015) The Use of Affinity Tags to Overcome Obstacles in Recombinant Protein Expression and Purification, Protein Pept Lett 22, 885-892.
[20] Guan, D, and Chen, Z. (2014) Challenges and recent advances in affinity purification of tag-free proteins, Biotechnol Lett 36, 1391-1406.
[21] Guzzo, C M, and Yang, D C. (2007) Systematic analysis of fusion and affinity tags using human aspartyl-tRNA synthetase expressed in E. coli, Protein Expr Purif 54, 166-175.
[22] Kimple, M E, Brill, A L, and Pasker, R L. (2013) Overview of affinity tags for protein purification, Curr Protoc Protein Sci 73, Unit 9 9.
[23] Li, Y. (2010) Commonly used tag combinations for tandem affinity purification, Biotechnol Appl Biochem 55, 73-83.
[24] Pina, A S, Batalha, I L, and Roque, A C. (2014) Affinity tags in protein purification and peptide enrichment: an overview, Methods Mol Biol 1129, 147-168.
[25] Sun, P, Tropea, J E, and Waugh, D S. (2011) Enhancing the solubility of recombinant proteins in Escherichia coli by using hexahistidine-tagged maltose-binding protein as a fusion partner, Methods Mol Biol 705, 259-274.
[26] Walls, D, and Loughran, S T. (2011) Tagging recombinant proteins to enhance solubility and aid purification, Methods Mol Biol 681, 151-175.
[27] Wingfield, P T. (2015) Overview of the purification of recombinant proteins, Curr Protoc Protein Sci 80, 6 1 1-35.
[28] Wood, D W. (2014) New trends and affinity tag designs for recombinant protein purification, Curr Opin Struct Biol 26, 54-61.
[29] Villaverde, A, and Carrio, M M. (2003) Protein aggregation in recombinant bacteria: biological role of inclusion bodies, Biotechnol Lett 25, 1385-1395.
[30] Stevens, R C. (2000) Design of high-throughput methods of protein production for structural biology, Structure 8, R177-185.
[31] Song, J M, An, Y J, Kang, M H, Lee, Y H, and Cha, S S. (2012) Cultivation at 6-10 degrees C. is an effective strategy to overcome the insolubility of recombinant proteins in Escherichia coli, Protein Expr Purif 82, 297-301.
[32] Sorensen, H P, and Mortensen, K K. (2005) Soluble expression of recombinant proteins in the cytoplasm of Escherichia coli, Microb Cell Fact 4, 1.
[33] Ohana, R F, and Hurst, R. (2015) Purification of Recombinant Proteins from Cultured Mammalian Cells by HaloTag Technology, Curr Protoc Mol Biol 110, 10 31 11-10 31 15.
[34] Zordan, R E, Beliveau, B J, Trow, J A, Craig, N L, and Cormack, B P. (2015) Avoiding the ends: internal epitope tagging of proteins using transposon tn7, Genetics 200, 47-58.
[35] Mooney, J T, Fredericks, D P, Christensen, T, Bruun Schiodt, C, and Hearn, M T. (2015) N-terminal processing of affinity-tagged recombinant proteins purified by IMAC procedures, J Mol Recognit.
[36] Liu, X, Huang, A, Luo, D, Liu, H, Han, H, Xu, Y, and Liang, P. (2015) Use of adenylate kinase as a solubility tag for high level expression of T4 DNA ligase in Escherichia coli, Protein Expr Purif 109, 79-84.
[37] Costa, S J, Almeida, A, Castro, A, Domingues, L, and Besir, H. (2013) The novel Fh8 and H fusion partners for soluble protein expression in Escherichia coli: a comparison with the traditional gene fusion technology, Appl Microbiol Biotechnol 97, 6779-6791.
[38] Porath, J, Carlsson, J, Olsson, I, and Belfrage, G. (1975) Metal chelate affinity chromatography, a new approach to protein fractionation, Nature 258, 598-599.
[39] Kuo, W H, and Chase, H A. (2011) Exploiting the interactions between poly-histidine fusion tags and immobilized metal ions, Biotechnol Lett 33, 1075-1084.
[40] Smith, D B, and Johnson, K S. (1988) Single-step purification of polypeptides expressed in Escherichia coli as fusions with glutathione S-transferase, Gene 67, 31-40.
[41] Boisselier, E, Audet, M L, Cantin, L, and Salesse, C. (2011) A strategy for purifying glutathione S-transferase in the presence of sodium dodecyl sulfate, Biotechniques 51, 193-194.
[42] Desmeules, P, Penney, S E, and Salesse, C. (2006) Single-step purification of myristoylated and nonmyristoylated recoverin and substrate dependence of myristoylation level, Anal Biochem 349, 25-32.
[43] Flaherty, K M, Zozulya, S, Stryer, L, and McKay, D B. (1993) Three-dimensional structure of recoverin, a calcium sensor in vision, Cell 75, 709-716.
[44] Tanaka, T, Ames, J B, Harvey, T S, Stryer, L, and Ikura, M. (1995) Sequestration of the membrane-targeting myristoyl group of recoverin in the calcium-free state, Nature 376, 444-447.
[45] Hughes, R E, Brzovic, P S, Klevit, R E, and Hurley, J B. (1995) Calcium-dependent solvation of the myristoyl group of recoverin, Biochemistry 34, 11410-11416.
[46] Ames, J B, Ishima, R, Tanaka, T, Gordon, J I, Stryer, L, and Ikura, M. (1997) Molecular mechanics of calcium-myristoyl switches, Nature 389, 198-202.
[47] Bussieres, S, Buffeteau, T, Desbat, B, Breton, R, and Salesse, C. (2008) Secondary structure of a truncated form of lecithin retinol acyltransferase in solution and evidence for its binding and hydrolytic action in monolayers, Biochim. Biophys. Acta 1778, 1324-1334.
[48] Horchani, H, Bussieres, S, Cantin, L, Lhor, M, Laliberte-Gemme, J S, Breton, R, and Salesse, C. (2014) Enzymatic activity of Lecithin:retinol acyltransferase: A thermostable and highly active enzyme with a likely mode of interfacial activation, Biochim Biophys Acta 1844, 1128-1136.
[49] Studier, F W. (2005) Protein production by auto-induction in high density shaking cultures, Protein Expr Purif 41, 207-234.
[50] Demers, E, Boisselier, E, Horchani, H, Blaudez, D, Calvez, P, Cantin, L, Belley, N, Champagne, S, Desbat, B, and Salesse, C. (2015) Lipid Selectivity, Orientation, and Extent of Membrane Binding of Nonacylated RP2, Biochemistry 54, 2560-2570.
[51] Laemmli, U K. (1970) Cleavage of structural proteins during the assembly of the head of bacteriophage T4, Nature 227, 680-685.
[52] Hunt, I. (2005) From gene to protein: a review of new and enabling technologies for multi-parallel protein expression, Protein Expr Purif 40, 1-22.
[53] di Guan, C, Li, P, Riggs, P D, and Inouye, H. (1988) Vectors that facilitate the expression and purification of foreign peptides in Escherichia coli by fusion to maltose-binding protein, Gene 67, 21-30.
[54] Kapust, R B, and Waugh, D S. (1999) Escherichia coli maltose-binding protein is uncommonly effective at promoting the solubility of polypeptides to which it is fused, Protein Sci 8, 1668-1674.
[55] Fox, J D, Routzahn, K M, Bucher, M H, and Waugh, D S. (2003) Maltodextrin-binding proteins from diverse bacteria and archaea are potent solubility enhancers, FEBS Lett 537, 53-57.
[56] Bucher, M H, Evdokimov, A G, and Waugh, D S. (2002) Differential effects of short affinity tags on the crystallization of Pyrococcus furiosus maltodextrin-binding protein, Acta Crystallogr D Biol Crystallogr 58, 392-397.
[57] Baneyx, F. (1999) Recombinant protein expression in Escherichia coli, Curr Opin Biotechnol 10, 411-421.
[58] Routzahn, K M, and Waugh, D S. (2002) Differential effects of supplementary affinity tags on the solubility of MBP fusion proteins, J Struct Funct Genomics 2, 83-92.
[59] LaVallie, E R, DiBlasio, E A, Kovacic, S, Grant, K L, Schendel, P F, and McCoy, J M. (1993) A thioredoxin gene fusion expression system that circumvents inclusion body formation in the E. coli cytoplasm, Biotechnology (NY) 11, 187-193.
[60] Panavas, T, Sanders, C, and Butt, T R. (2009) SUMO fusion technology for enhanced protein production in prokaryotic and eukaryotic expression systems, Methods Mol Biol 497, 303-317.
[61] Bourne e al. (2001) J Biol Chem 276: 11949-11955).
[62] Ames et al. (2000) Biochemistry 39: 12149-12161) (PMID: 11015193).
[63] Hanson et al. (2014) BMC Bioinformatics 15: 87 (PMID: 24669788).

SEQUENCE LISTING

SEQ ID NO. 1
Nucleic acid sequence-full length native Recoverin
ATGGGGAACAGCAAGAGTGGGGCCCTGTCCAAGGAGATCCTGGAGGAGCTGCAGCTG
AACACCAAGTTCACGGAGGAGGAGCTGAGCTCCTGGTACCAGTCCTTCCTGAAGGAGT
GCCCCAGTGGCCGGATCACCCGGCAGGAGTTCCAGACCATCTACTCCAAGTTCTTCCC
CGAGGCCGACCCCAAGGCCTATGCCCAGCACGTGTTCCGAAGCTTTGATGCCAACAG
CGATGGCACCTTGGACTTCAAGGAGTATGTCATCGCCCTACACATGACCAGCGCGGGC
AAGACCAACCAGAAGCTGGAGTGGGCCTTCTCCCTCTATGATGTGGATGGCAATGGGA
CCATCAGCAAGAACGAGGTGCTGGAGATTGTCACGGCTATCTTCAAAATGATCAGCCC
TGAGGACACAAAGCATCTCCCAGAAGACGAGAACACTCCGGAAAAGCGAGCAGAGAA
GATCTGGGGATTCTTTGGCAAGAAGGATGATGATAAACTTACAGAGAAAGAATTCATCG
AAGGGACCCTGGCCAATAAGGAAATTCTGCGACTGATTCAATTCGAGCCTCAAAAAGT
GAAGGAGAAACTGAAGGAAAAGAAACTCTGA (codon stop)
SEQ ID NO. 2
Amino acid sequence-full length native Recoverin (start Met is removed)
GNSKSGALS KEILEELQLN TKFTEEELSS WYQSFLKECP SGRITRQEFQ TIYSKFFPEA
DPKAYAQHVF RSFDANSDGT LDFKEYVIAL HMTSAGKTNQ KLEWAFSLYD
VDGNGTISKN EVLEIVTAIF KMISPEDTKH LPEDENTPEK RAEKIWGFFG KKDDDKLTEK
EFIEGTLANK EILRLIQFEP QKVKEKLKEK KL
SEQ ID NO. 3
Nucleic acid sequence-mutated Recoverin (+ Ndel and BamHI restriction
sites, + silent mutation, without myristoyl group)
Nde1 Site
GGGAACAGCAAGAGTGGGGCCCTGTCCAAGGAGATCCTGGAGGAGCTGCAG
CTGAACACCAAGTTCACGGAGGAGGAGCTGAGCTCCTGGTACCAGTCCTTCCTGAAGG
AGTGCCCCAGTGGCCGGATCACCCGGCAGGAGTTCCAGACCATCTACTCCAAGTTCTT
CCCCGAGGCCGACCCCAAGGCCTATGCCCAGCACGTGTTCCGAAGCTTTGATGCCAA
CAGCGATGGCACCTTGGACTTCAAGGAGTATGTCATCGCCCTACACATGACCAGCGCG
GGCAAGACCAACCAGAAGCTGGAGTGGGCCTTCTCCCTCTATGACGTGGATGGCAAT
GGGACCATCAGCAAGAACGAGGTGCTGGAGATTGTCACGGCTATCTTCAAAATGATCA
GCCCTGAGGACACAAAGCATCTCCCAGAAGACGAGAACACTCCGGAAAAGCGAGCAG
AGAAGATCTGGGGATTCTTTGGCAAGAAGGATGATGATAAACTTACAGAGAAAGAGTTC
ATCGAAGGGACCCTGGCCAATAAGGAAATTCTGCGACTGATTCAATTCGAGCCTCAAAA
AGTGAAGGAGAAACTGAAGGAAAAGAAACTC GGC GGT GGT GGC GGC CTG GTT
CCG CGT GGATCC
T to C : silent mutation
Eco R1 site changed GAATTC for GAGTTC: silent mutation
5 glycins (GGC ou GGT) at the end + thrombin cleavage site (Leu-Val-Pro-Arg-Gly-Ser:
L (CTG), V (GTT), P (CCG), R (CGT), G (GGA), S (TCC))
Stop codon removed and replaced by BamH1 site (GGATTC) that corresponds also to glycine
and serine from thrombin recognition site
SEQ ID NO. 4
Amino acid sequence-mutated Recoverin (+ NdeI and BamHI restriction sites, + silent
mutation, without myristoyl group)
GNSKSGALS KEILEELQLN TKFTEEELSS WYQSFLKECP SGRITRQEFQ TIYSKFFPEA
DPKAYAQHVF RSFDANSDGT LDFKEYVIAL HMTSAGKTNQ KLEWAFSLYD
VDGNGTISKN EVLEIVTAIF KMISPEDTKH LPEDENTPEK RAEKIWGFFG KKDDDKLTEK
EFIEGTLANK EILRLIQFEP QKVKEKLKEK KLGGGGGLVPRGS
(added sequence): GGGGGLVPRGS

1. A polynucleotide molecule comprising a sequence encoding a protein of interest to be purified, and a tag polynucleotide encoding a protein of an EF-hand calcium-binding family of proteins, wherein said EF-hand calcium-binding protein undergoes conformational change under presence or absence of calcium wherein the EF-hand calcium-binding protein is a neuronal calcium sensor protein selected from the group consisting of: Recoverin, GCAP1, GCAP2, GCAP3, GCIP, KChIP1, KChIP2, Calsenilin/DREAM, NCS1/Frequenin, Neurocalcin delta, Hippocalcin, and VILIP.

2.-7. (canceled)

8. The polynucleotide of claim 1, wherein the tag polynucleotide encodes Recoverin.

9. The polynucleotide of claim 8, wherein said encoded Recoverin tag is substantially full length.

10. The polynucleotide of claim 9, wherein said encoded Recoverin tag has a molecular weight of about 23 kD.

11. The polynucleotide according to claim 8, wherein said encoded Recoverin is defined by SEQ ID NO. 2 or 4.

12. The polynucleotide of claim 1, wherein the tag polynucleotide further encodes a proteolytic-cleavage site at a terminal end of the tag protein.

13. (canceled)

14. The polynucleotide of claim 1, wherein the protease is selected from the group consisting of: thrombin, TEV and HRV C3.

15. (canceled)

16. The polynucleotide of claim 1, wherein the tag polynucleotide has about 90% or more nucleic acid identity to SEQ ID NO. 1.

17. The polynucleotide of claim 16, defined by SEQ ID NO. 3

18. The polynucleotide according to claim 1, having introduced therein at, or near, one of its N- or C-terminus, one or more sequence encoding a restriction site.

19. (canceled)

20. The polynucleotide of claim 19, wherein said protein of interest is located at the C-terminus of the tag molecule.

21.-25. (canceled)

26. A method for expressing a protein of interest, comprising the steps of:
expressing said protein of interest fused to a Recoverin tag (TagR) in an expression system comprising a vector, an expression cassette or a host cell, all of which comprising a polynucleotide of claim 1.

27.-28. (canceled)

29. The method of claim 26, further comprising the step of:
producing a protein of interest by:
growing said host cell under conditions that permit expression of the protein according to claim 26.

30. The method of claim 29, further comprising the steps of:
purifying said fused-protein by separating said fused protein from unwanted components on hydrophobic affinity chromatography in presence of calcium.

31. The method of claim 30, wherein said purifying step is carried out by eluting said fused-protein from said hydrophobic affinity column in presence of a calcium chelator; and optionally cleaving said eluted fused molecule in order to obtain the purified protein of interest cleaved from the tag.

32. The method of claim 30, wherein said purifying step is carried out by cleaving said fused-protein of interest from said tag to obtain a mixture comprising said cleaved protein and the tag, and separating said cleaved protein of interest from said tag by eluting said cleaved protein on said hydrophobic affinity column in presence of a calcium chelator.

33.-35. (canceled)

36. A fusion protein encoded by the polynucleotide according to claim 1.

37.-41. (canceled)

42. The protein of claim 36, wherein the encoded protein tag is nonmyristoylated.

43. (canceled)

44. The protein of claim 42, comprising a further Recoverin tag (TagR).

45. The protein of claim 44, comprising two molecules of Recoverin positioned at N-terminal position.

46.-49. (canceled)
